(19) United States
(12) Patent Application Publication
(10) Pub. No.: US 2017/0241966 A1
(43) Pub. Date: Aug. 24, 2017

(54) METHOD AND SYSTEM FOR DETERMINING THE FRACTIONS OF A STREAMING GASEOUS MEDIUM

(71) Applicant: BERKIN B.V., Ruurlo (NL)

(72) Inventors: Joost Conrad LÖTTERS, Ruurlo (NL); Jarno GROENESTEIJN, Ruurlo (NL); Theodorus Simon Josef LAMMERINK, Ruurlo (NL); Remco John WIEGERINK, Ruurlo (US); Egbert Jan VAN DER WOUDEN, Ruurlo (NL); Wouter SPARREBOOM, Ruurlo (NL)

(21) Appl. No.: 15/517,913

(22) PCT Filed: Oct. 5, 2015
(86) PCT No.: PCT/NL2015/050698

§ 371 Date: Apr. 7, 2017

(30) Foreign Application Priority Data
Oct. 7, 2014 (NL) 2013587

(51) Int. Cl. G01N 33/00

(52) U.S. Cl. CPC G01N 33/0036

(57) ABSTRACT

The invention relates to a method and a system for determining the fractions of a flowing gaseous medium that comprises a known plurality N of known components. The method comprises the steps for determining at least N−1 parameters of a flowing gaseous medium. The N−1 parameters are chosen from a group of quantities comprising mass flow, density, viscosity, and heat capacity. At least N−1 reference values are provided for each of the known N components relating to each of the determined N−1 quantities. The fraction of each of the known components of the supplied gaseous medium is determined through solving of at least N equations. The N equations comprise N−1 equations which describe each determined parameter as a function of the fraction and the reference values, plus an equation that sets the sum of the fractions so as to be equal to 100%.

The invention relates to a method of determining the fractions of a flowing gaseous medium. The invention further relates to a system for implementing such a method.

A knowledge of the composition of a flowing gas is important in many fields of technology. This is the case, for example, in the manufacture of medicines or in composing a desired mixture of gases for medical purposes.

It is essential, for example, in medical infusion pumps, especially in the field of neonatology, to know both the flow rate and the composition of the medium, for example if a newly born child is to receive both the correct kind and the correct quantity of drugs and/or nutrients. A problem here is that the flow rates are very low, which renders it difficult to achieve the desired accuracy of the measurements.

But it is equally important in the case of natural gas to know the composition thereof, for example for determining its energy content. Conventional devices for determining the energy content of gaseous fuels, such as a Wobbe index meter or a gas chromatograph, are comparatively bulky and expensive.

It is expected that the composition and quality of the natural gas in the national grid will vary substantially as a result of the mixing of natural gases from various countries and the periodic variations that will occur as a result thereof. Quality control and quality assurance are of major importance in this respect. This is even more relevant as it is desirable also to introduce biogas into the national grids.

It is apparent from the above that there is a need for a fast, inexpensive and reliable method of determining the composition of a gaseous medium in a plurality of technological fields. It is accordingly an object of the present invention to provide a method by which the (volume) fractions of a flowing gaseous medium can be determined and by which a determination of said fractions can take place continuously (in real time), in particular for purposes of quality assurance and safety.

To achieve this object, the present invention provides a method as defined in claim 1. The method of determining the fractions, in particular the volume fractions according to the present invention, comprises a step of providing the flowing gaseous medium of which the composition is to be determined. The flowing gaseous medium consists at least substantially of a known plurality N of known components. The term “components” herein denotes in any case pure, unmixed fluids such as, for example, water, hydrogen, oxygen, carbon dioxide, nitrogen, and alkanes such as methane, ethane, propane, etc.

According to the method, at least N−1 parameters are determined of the gaseous medium provided. In an embodiment, for example, one or more of said parameters are chosen from a group of quantities comprising mass flow, density, viscosity, and heat capacity. Alternative quantities are obviously conceivable. The parameters may be directly measured, or alternatively be derived from other measurements.

For each of the N known components, at least N−1 reference values are provided relating to each of the determined N−1 quantities. In other words, a reference value is provided for each of the known components of the gaseous medium. If, for example, the density of a mixture of methane, carbon dioxide and nitrogen is determined or measured, the respective densities of methane, carbon dioxide and nitrogen are provided as the reference values. If supplementary parameters are measured such as, for example, the viscosity, a reference value for the determined quantity is provided, so in this case the viscosity, for each of the components.

The method according to the present invention comprises a step of determining the fraction of each of the known components of the provided gaseous medium through solving of at least N equations, which equations comprise:
at least N−1 equations which describe each determined parameter as a function of the fraction of each of the known parameters of the medium and as a function of the provided reference values of each of the known components of the gaseous medium, and
at least one equation which sets the sum of the fractions of each of the known components at least substantially so as to be equal to 100%.

The above method renders it possible to determine the composition of a flowing gas in a comparatively simple and fast manner. Solving the equations according to the invention leads substantially instantaneously to a determination of the composition. This renders possible in particular a continuous monitoring (in real time) of the flowing gaseous medium. The object of the present invention is achieved thereby.

Advantageous embodiments of the method are defined in the dependent claims 2 to 12. The advantages of these embodiments will be discussed below.

In an embodiment, the method comprises a step of substantially continuously providing the flowing gaseous medium and of substantially continuously determining the at least N−1 parameters. The method can thus be carried out substantially continuously for determining the fractions of the flowing gaseous medium substantially in real time. The steps of determining the parameters and of determining the fractions of the components are repeated at least once, so that the composition of the continuously flowing gaseous medium is known at two moments in time. This renders it possible to view the composition over time, so that the quality of the gas can be monitored. This enhances the safety aspect, in particular in medical applications.

The method according to the present invention yields very quick results through the determination of the N−1 parameters and solving of the N equations. Compared with alternative, known methods such as, for example, gas chromatography, wherein a result of the measurement becomes available after approximately 3 minutes, the method according to the present invention renders possible a very quick result of the order of 0 to 60 seconds, in particular 0 to 15 seconds, more in particular 0 to 5 seconds. In addition, the gas can be provided according to the present invention without the necessity of a pre-treatment (for example a separation of components and/or the addition of a carrier gas, as in gas chromatography). This absence of a pre-treatment and the speed that is achievable with the method render it possible for the method to be used continuously or semi-continuously. This is particularly advantageous in situations where monitoring of the gas is necessary or desirable.

In an embodiment, the equations are described in a matrix equation which is subsequently solved. An efficient, fast and reliable method of solving such a matrix equation is the method of least squares, which is known per se. A processing unit is preferably used for solving the matrix equation so as to obtain the fractions of the components.

In an embodiment, it is an undesired component in the gaseous medium that is monitored. Thus, for example, the presence of oxygen or hydrogen in a gaseous medium may be detected. In that case it is stipulated that the gaseous medium contains the relevant component even though the initial fraction of said component is equal to zero. The method according to the present invention thus also expressly relates to those situations in which one of the known components is not yet present in the gas, but wherein this known component may be present in the future. In other words, the fraction of the known component may be equal to zero.

The method according to the present invention is particularly suitable for determining the fractions of a flowing gaseous medium that substantially comprises three or four known components, although it can also be applied to the presence of more than four components in principle. The above expression “substantially comprises three or four known components” is meant to indicate that the sum of the fractions of said three or four components is substantially equal to 100%. It is conceivable that a further known or unknown component is present in the gas, which further component accounts for only a tiny portion of the total fraction. Such a component may be present, for example, in a concentration lower than 5%, preferably lower than 2%, particularly lower than 1%. In such a case the method comprises a step of disregarding this further component in the equations.

It is conceivable that one of the known components is CH₄, C₃H₈ N₂, and/or CO₂, especially in the case of natural gas or similar gases. It is furthermore conceivable that one of the known components is O₂ or H₂. Other compositions, however, comprising known components are also possible.

In an embodiment, the method according to the present invention comprises a step of determining two parameters, in particular the density and the heat capacity of the gaseous medium. The determination of two parameters is suitable for determining the fractions of a gaseous medium having three known components.

The two parameters may be determined by means of signals from a thermal flow sensor and a flow sensor of the Coriolis type.

In an embodiment of the method, furthermore, a measure for the calorific value of the flowing gaseous medium is derived from the fractions thus determined.

In a further embodiment, the Wobbe index WI of the gaseous medium is determined from the calorific value as follows:

$$WI = \frac{H}{\sqrt{{G}_{s}}}$$

wherein H (J/m³) is the amount of thermal energy generated by complete combustion of a given volume of the medium comprising a gas mixture and air, and Gₛ (-) is the ratio of mass densities of the gas mixture and air. The composition of the medium is determined by a system according to the present invention with a high accuracy such that the Wobbe index can be accurately determined, for example in accordance with the above equation.

It is furthermore conceivable that the method comprises a step of controlling the mass flow of the flowing gaseous medium on the basis of the determined fractions thereof. It is possible here that the control comprises a step of completely reducing the mass flow to zero, for example upon detection of an undesired component. It is furthermore conceivable that the method comprises a step of issuing a warning signal when one or several of the determined fractions is or are higher or lower than a preset standard value.

According to an aspect, the invention provides a system whereby the method can be implemented, said system being defined in claim 13. The system according to the present invention comprises a flow tube having an inlet and an outlet for supplying and discharging the flowing gaseous medium, respectively, in particular in a continuous manner, the composition of said medium having to be determined. Sensor means are provided for determining the at least N−1 parameters of the supplied gaseous medium. Said sensor means are preferably connected to the flow tube or form part thereof. The system further comprises a processing unit that is connected to the sensor means, which processing unit has the at least N−1 reference values stored therein and is designed for determining the fraction of each of the known components of the supplied gaseous medium by solving the at least N equations.

The system according to the present invention is thus designed for determining a composition of a gaseous medium that is a mixture of N known components. The processing unit contains N equations which describe the respective quantities associated with the at least N−1 parameters as a function of fractions of the N components in the medium.

First of all, the processing unit contains the equation which describes the sum of the fractions of the components of the medium as being equal to 100%, or at least substantially equal to 100%. In addition, the processing unit contains N−1 equations for the at least N−1 quantities determined by the sensor means as a function of the fractions of the components. Thus, for example, the density and the viscosity may both be stored as linear functions of the components in the form of equations in the processing unit.

There are N equations in N unknowns present in the processing unit in this manner. The processing unit is designed for solving these equations so as to obtain the fraction of each of the components. Methods of solving a number of equations with the same number of unknowns are known per se.

Advantageous embodiments of the system are defined in the dependent claims 14 to 20. The advantages of these and other embodiments will be explained below.

In an embodiment, the sensor means and the processing unit are designed for determining the N−1 parameters and fractions in a repetitive manner, in particular continuously. This is to say that the parameters and the fractions of the supplied gas can be determined substantially continuously/semi-continuously/intermittently.

The system may be designed, for example, for determining the fractions repetitively with time intervals that lie between 0 and 60 seconds, in particular between 0 and 15 seconds, more in particular between 0 and 5 seconds. This renders the system many times faster than the systems known at present such as, for example, gas chromatography. In an embodiment, the processing unit is provided with a reference table or database in which the reference values are stored. Such reference tables and databases are generally known and comprise values for the properties and parameters such as the density, viscosity and specific heat capacity of known fluids. The processing unit can compare these data with the parameters determined for the medium. The processing unit can then determine the fractions of the known components using the stored equations for the parameters. It is conceivable for the processing unit to be designed for comparing, fitting, or interpolating. This simplifies and speeds up the solving of the equations.

The sensor means in an embodiment comprise at least one of the following: a density sensor, a flow sensor of the Coriolis type, a thermal flow sensor, and/or a pressure sensor. The pressure sensor may be, for example, a differential pressure sensor, and the flow sensor of the Coriolis type may at the same time form the pressure sensor in an embodiment. The processing unit is preferably constructed such in this case that it furthermore determines by means of calculations or modelling one or several of the following: viscosity, specific heat capacity, and thermal conductivity, from the parameters measured by the sensors mentioned above. The sensors send a signal to the processing unit. It is possible in this respect that signal processing means are provided for processing the signal, for example through noise reduction, signal corrections, or mathematical operations such as integration and/or transforms.

In a special embodiment which is comparatively inexpensive, small, and efficient, the sensor means comprise each of the following: a density sensor, a flow sensor of the Coriolis type, a thermal flow sensor, and a pressure sensor. Such sensors are commercially available, for example under the designations Avenisens, Bronkhorst Cori-Tech M13, Bronkhorst EL-flow and Bronkhorst EL-press. Other brands and/or types of sensors are obviously conceivable.

In a practical embodiment in which the sensor means comprise at least a thermal flow sensor and a flow sensor of the Coriolis type, the processing unit is designed for determining the specific heat capacity of the medium based on signals coming both from the thermal flow sensor and from the flow sensor of the Coriolis type. Applicant's Dutch Patent Application NL 2 012 126, which document is to be deemed fully included in the present Application by reference, describes how the specific heat capacity of a medium can be determined from the slope of a signal of the thermal flow sensor plotted against a signal from the flow sensor of the Coriolis type, as is also described in Lötters, J. C. et al., 2014, Integrated multi-parameter flow measurement system, in 2014 IEEE 27th International Conference on Micro Electro Mechanical Systems (MEMS) [DOI: 10.1109/MEMSYS.2014.6765806].

In an embodiment in which the sensor means comprise at least a flow sensor of the Coriolis type and a pressure sensor, the processing unit is designed for determining the viscosity of the medium based on signals both from the flow sensor of the Coriolis type and from the pressure sensor. The cited NL 2 012 126 describes how the viscosity of the medium can be determined from the slope of the signal from the Coriolis type flow sensor plotted against a signal from the pressure sensor. This is again described in Lötters, J. C. et al., 2014, Integrated multi-parameter flow measurement system, in 2014 IEEE 27th International Conference on Micro Electro Mechanical Systems (MEMS) [DOI: 10.1109/MEMSYS.2014.6765806].

In an embodiment in which the sensor means comprise at least a pressure sensor and a thermal flow sensor, the pressure sensor is arranged such that it determines a differential pressure across the thermal flow sensor.

A number of examples of the use of equations for determining the fractions of the components are given below.

### EXAMPLE 1

In a first example, the three (volume) fractions are indicated by φᵢ for the determination of a medium with three components, which gives the following equation (in which N=3):

$${\sum }_{i = 1}^{N} {ϕ}_{i} = 1 \qquad ( 1 )$$

According to the method, N−1=2 parameters are to be measured or derived then. These at least two parameters of the medium may be, for example, the density ρ and the viscosity η of the medium. The density and viscosity of the medium are a function of the fractions of the known components and the density and viscosity of the relevant known component.

The density and viscosity of each component being denoted ρᵢ and ηᵢ, respectively, we get the following dependencies:

$${\sum }_{i = 1}^{N} {ϕ}_{i} {ρ}_{i} = ρ \\ {\sum }_{i = 1}^{N} {ϕ}_{i} {η}_{i} = η \qquad ( 2 )$$

These dependencies may be written as a matrix equation:

$$( \begin{matrix}{ρ}_{1} & {ρ}_{2} & {ρ}_{3} \\ {η}_{1} & {η}_{2} & {η}_{3} \\ 1 & 1 & 1\end{matrix} ) \cdot ( \begin{matrix}{ϕ}_{1} \\ {ϕ}_{2} \\ {ϕ}_{3}\end{matrix} ) = ( \begin{matrix}ρ \\ η \\ 1\end{matrix} ) \qquad ( 3 )$$

The density ρᵢ and the viscosity ηᵢ of each component are stored in the processing unit in an embodiment, for example in a reference table, and the density ρ and the viscosity η of the medium are measured.

Since only the fractions φᵢ are unknown, this leads to a set of three equations in three unknowns. This set can be solved so as to determine the values of the fractions φᵢ, for example by inverting the matrix. Alternative combinations of parameters other than those described above, such as density and specific heat capacity or viscosity and specific heat capacity, are equally conceivable. It is furthermore possible for the group of at least two parameters to include thermal conductivity.

### EXAMPLE 2

For determining the composition of a medium with four known components, an embodiment comprises the determination of an additional component. According to the method, three parameters of the medium are determined then. The medium is a mixture of four components here, the three parameters of the medium being dependent on the fractions of the components.

The specific heat capacity cₚ of the medium is additionally determined in this example. The specific heat capacity of each of the components being denoted cₚᵢ, we get the following matrix equation:

$$( \begin{matrix}{ρ}_{1} & {ρ}_{2} & {ρ}_{3} & {ρ}_{4} \\ {η}_{1} & {η}_{2} & {η}_{3} & {η}_{4} \\ 1 & 1 & 1 & 1 \\ {ρ}_{1}^{*} {c}_{p 1} & {ρ}_{2}^{*} {c}_{p 2} & {ρ}_{3}^{*} {c}_{p 3} & {ρ}_{4}^{*} {c}_{p 4}\end{matrix} ) \cdot ( \begin{matrix}{ϕ}_{1} \\ {ϕ}_{2} \\ {ϕ}_{3} \\ {ϕ}_{4}\end{matrix} ) = ( \begin{matrix}ρ \\ η \\ 1 \\ {ρ}^{*} {20}_{p}\end{matrix} ) \qquad ( 4 )$$

This equation can be solved so as to obtain values for the four unknowns, i.e. the fractions φᵢ, whereby the composition of the mixture of four components is determined.

Following the principle outlined above, it is possible to determine the composition of a medium having five components by determining a further parameter which is dependent on the fractions of the components, such as the thermal conductivity. This principle may be extended to a medium having N components, in which case N−1 parameters are to be determined. It is also conceivable that the fractions are determined by methods other than the solving of equations as described above, for example by fitting or interpolating of parameters.

The invention will be explained in more detail below with reference to the appended figures, in which:

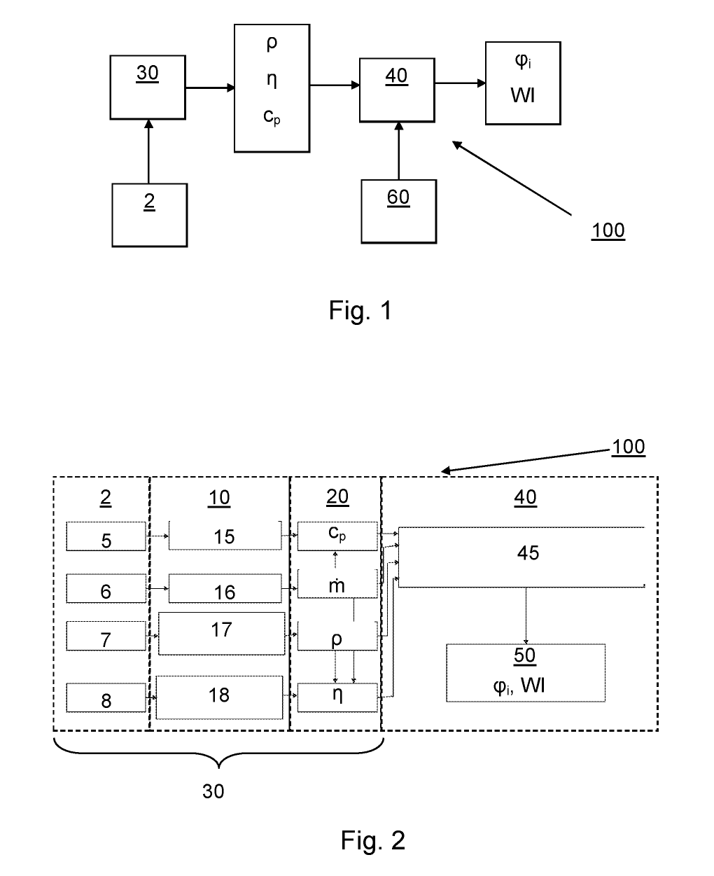

FIG. 1 diagrammatically shows a system according to the present invention;

FIG. 2 diagrammatically shows a system having a plurality of sensors according to the present invention;

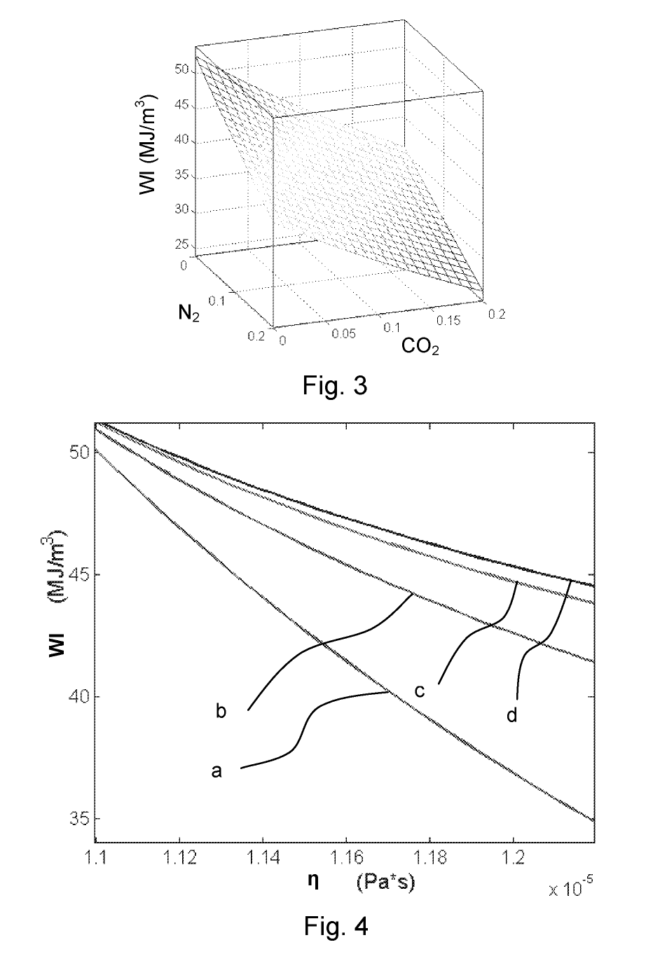

FIG. 3 shows the dependence of the Wobbe index of a biogas as a function of CO₂ and N₂;

FIG. 4 is a graph showing the Wobbe index WI as a function of the viscosity η; and

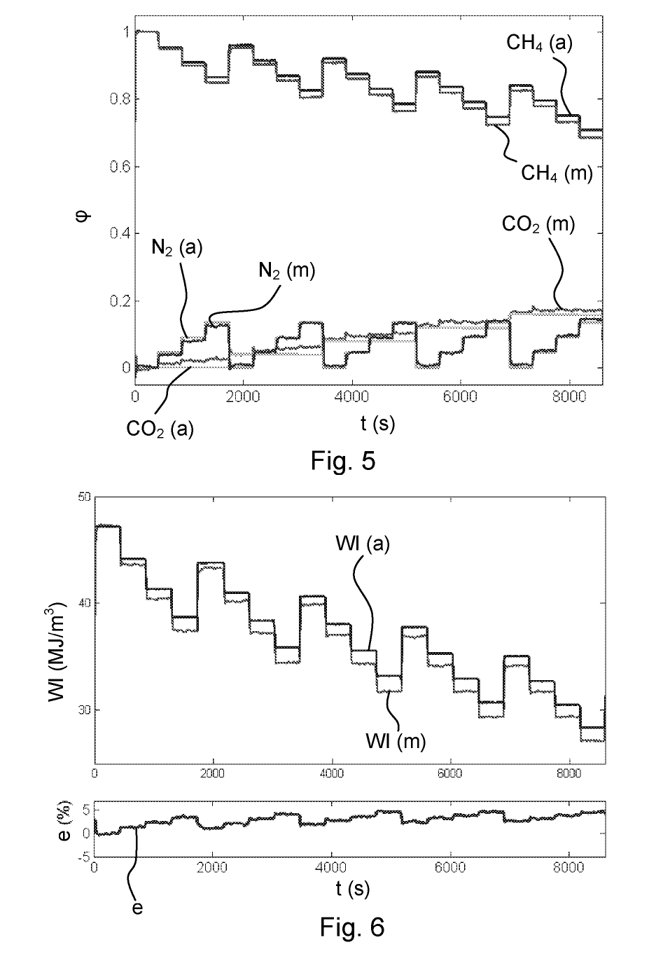

FIGS. 5 to 8 show results of measurements carried out by a system according to the present invention.

FIG. 1 diagrammatically shows a system 100 according to the present invention with which fractions of a flowing gaseous medium, which comprises at least substantially a known plurality N of known components, can be determined. The system 100 comprises a flow tube 2 for the medium of which the fractions are to be determined. The system comprises sensor means 30 which are connected to the flow tube 2 or which form part thereof. The sensor means 30 are designed for determining at least N−1 parameters of the medium. Said parameters are chosen from a group comprising density, viscosity, and specific heat capacity, indicated with the respective symbols ρ, η and cₚ in FIG. 1. The system is further provided with a processing unit 40 which is connected to the sensor means 30 and which is designed for determining the fraction of each of the components on the basis of the measured and/or determined parameters. The processing unit 40 in the embodiment shown is provided with a reference table 60 or database 60, shown schematically in FIG. 1, in which reference values for the measured and/or determined parameters of the known components are stored.

The operation of the system 100 will be explained below. The gaseous medium with the known components is conducted through the flow tube 2. The sensor means 30 are used for determining the at least N−1 parameters, either in that direct measurements are carried out, or in that the relevant parameters are determined on the basis of signals from the sensor means 30. It is alternatively possible that the signals are directly fed to the processing unit 40, where the parameters are determined. The processing unit 40 of FIG. 1 is designed for utilizing the data from the reference table 60 for determining the composition of the medium 2, for example by comparing the at least two parameters of the medium 2 with data from the reference table 60. The reference table 60 preferably also comprises information on the dependencies between the at least two parameters and the respective fractions pi of the components, for example in the form of formulae or functions.

The processing unit 40 of FIG. 1 comprises equations wherein are present on the one hand the at least two parameters of the medium and on the other hand the fractions of the components and the associated data from the reference table 60, such as the equations (1), (2), (3), and (4) described above. In other words: each of the at least N−1 parameters, for example ρ, η, and/or cₚ, of the medium is a function of the fraction φᵢ of the respective component and the associated data in the reference table 60. The processing unit 40 of FIG. 1 is capable of solving this set of equations for the N−1 parameters.

In an embodiment, the processing unit is designed for determining the fractions of the components in real time, i.e. substantially instantaneously. To achieve this, the set of equations may be arranged in the form of a matrix equation such as (3) or (4) for a simple and fast solution thereof by the processing unit 40.

In an embodiment, the processing unit 40 is designed also to determine a calorific value of the medium. It is possible in particular to determine the Wobbe index WI of the medium. The Wobbe index can be calculated from the fractions of the medium in combination with data from the reference table 60 by means of the equation mentioned above.

FIG. 2 diagrammatically shows a system 100 according to the present invention with sensor means 30 comprising sensors 5, 6, 7, and 8 which are provided on or adjacent to the flow tube 2. Said sensor means 30 in particular comprise a thermal flow sensor 5, a flow sensor of the Coriolis type 6, a density sensor 7, and a pressure sensor 8.

The sensor means 30 of FIG. 2 comprise a sensor processing unit 10. The latter is provided with a number of calculation models 15, 16, 17, 18 with which a plurality of parameters, comprising the specific heat capacity cₚ, the mass flow rate m, the density ρ, and the viscosity η of the medium, can be determined on the basis of the signals of the sensors 5, 6, 7, 8. Applicant's NL 2 012 126 cited above describes in great detail how the plurality of parameters can be determined by means of the sensors 5, 6, 7, and 8 mentioned above, as does Lötters, J. C. et al., 2014, Integrated multi-parameter flow measurement system, in 2014 IEEE 27th International Conference on Micro Electro Mechanical Systems (MEMS) [DOI: 10.1109/MEMSYS.2014.6765806]. A short clarification will be given below for the sake of brevity.

The output signal of the thermal flow sensor 5 is a measure for the flow rate and the heat capacity of the gas mixture. The pressure drop across the thermal flow sensor 5 is measured by the pressure sensor 8, which in particular is a differential pressure sensor 8. The output signal of the flow sensor of the Coriolis type 6 provides the mass flow rate, and the density is obtained from the density meter 7.

By comparing the output signals of the flow sensor of the Coriolis type 6 and the pressure sensor 8, taking into account the density, it is possible to calculate the viscosity.

Comparing the output signals of the thermal flow sensor 5 and the flow sensor of the Coriolis type 6 renders it possible to calculate the heat capacity of the gaseous medium.

The one or more parameters 20 thus obtained are fed to the equations 45 stored in the processing unit 40. The fractions (pi of the components, and preferably also the Wobbe index WI, can be determined in that the set of equations 45 is solved.

The processing unit 40 is designed, for example, for drawing up a matrix equation 45 such as described with reference to the equations (3) and (4). The processing unit completes the vector for the values of the parameters of the medium with the values determined by the assembly of sensors 1 and transmitted to the processing unit 40 via the parameter output 20. The quantities of the components, with the exception of the fractions are derived from a reference table 60 by the processing unit 40 and entered in the equations 45. The processing unit 40 subsequently solves the set of equations 45, as a result of which the fractions of the components of the medium are determined.

It is conceivable for a flow measuring system according to the cited Dutch Patent Application NL 2 012 126 to be connected to a processing unit according to the present invention so as to form a system according to the present invention. In an embodiment, the sensor signal processing unit 10 is integral with the processing unit 40.

FIG. 3 shows the dependence of the Wobbe index of a gas on the CO₂ and N₂ fractions. Such a gas may be, for example, a natural gas that is supplied to the gas grid. FIG. 3 shows the dependence of the Wobbe index on the nitrogen and carbon dioxide contents of the gas. Given such a strong variation in the composition of the gas mixture, an accurate and quick determination of that composition is desirable.

FIG. 4 is a graph showing the Wobbe index WI on the vertical axis as a function of the viscosity on the horizontal axis. It was found that there is a strong correlation between the viscosity and the Wobbe index if CO₂ is the only inert gas in the mixture. If there is also N₂ present, however, the correlation becomes less strong owing to the higher viscosity. This leads to a comparatively wide range within which the actual Wobbe index is situated. This range within which the Wobbe index may be situated is indicated by a lower index limit a and an upper index limit d in FIG. 4.

The method and the system according to the present invention render it possible to distinguish between CO₂ and N₂ by taking into account the density of the gas mixture, so that the range within which the actual value of the Wobbe index may lie can be narrowed so as to lie between a corrected lower index limit b and a corrected upper index limit c. According to the present invention, the determination of the Wobbe index becomes more accurate in that more than one parameter of the medium are determined, and the composition and thus the Wobbe index are determined on the basis thereof.

The FIGS. 5 to 8 show further results of measurements with a system according to the present invention. Methane, propane, carbon dioxide and nitrogen were added in quantities of the order of approximately 500 mlₙ/min to the system at a pressure of the order of 1.5 bar (absolute pressure). The output signals of the density sensor, pressure sensor, thermal sensor and the flow sensor of the Coriolis type were recorded during the measurements and processed by the method according to the present invention.

FIG. 5 shows the determination of the composition of a gas mixture with CH₄, CO₂ and N_{2.} Known quantities were supplied to the system. The known values of the added fractions (the so-termed applied fractions) are plotted against time tin FIG. 5: the applied CH₄ fraction CH₄ (a), the applied CO₂ fraction CO₂ (a) and the applied N₂ fraction N₂ (a). The applied fractions “(a)” are set, for example by means of a flowmeter, and vary in time step by step, as can be seen in the square waveforms of the applied fractions CH₄ (a), CO₂ (a), and N₂ (a). The values measured by a system according to the present invention are denoted “(m)”. The measured CH₄ fraction CH₄ (m), the measured CO₂ fraction CO₂ (m), and the measured N₂ fraction N₂ (m) are plotted against time t in FIG. 5. It is apparent from FIG. 5 that the values of the fractions CH₄ (m), CO₂ (m), and N₂ (m) as determined by a system according to the present invention follow the applied, i.e. actual fractions CH₄ (a), CO₂ (a), and N₂ (a) in real time. The values of the measured fractions CH₄ (m), CO₂ (m), and N₂ (m) lie within 5 percent of the applied values CH₄ (a), CO₂ (a), and N₂ (a). The system according to the present invention is thus not only fast, but also accurate.

FIG. 6 shows the determination of the Wobbe index of the gas mixture of FIG. 5. Since the composition of the gas mixture is known, as is its density, the Wobbe index can be calculated. The applied Wobbe index of the gas mixture is denoted WI (a). It is apparent from FIG. 6 that the Wobbe index varies stepwise in time. A system according to the present invention thus determines the Wobbe index of the gas mixture, the relevant values of which are denoted WI (m). FIG. 6 shows that the curve of the determined Wobbe index follows the curve of th e applied, i.e. actual Wobbe index WI (a). A change in the applied value of the Wobbe index is followed substantially instantaneously by an adaptation of the determined Wobbe index WI (m). The deviation e is plotted in the lower part of FIG. 6. The determined values WI (m) lie within a deviation range of five percent with respect to the applied values WI (a). The system according to the present invention is accordingly designed for an instantaneous and accurate determination of the Wobbe index values WI (a).

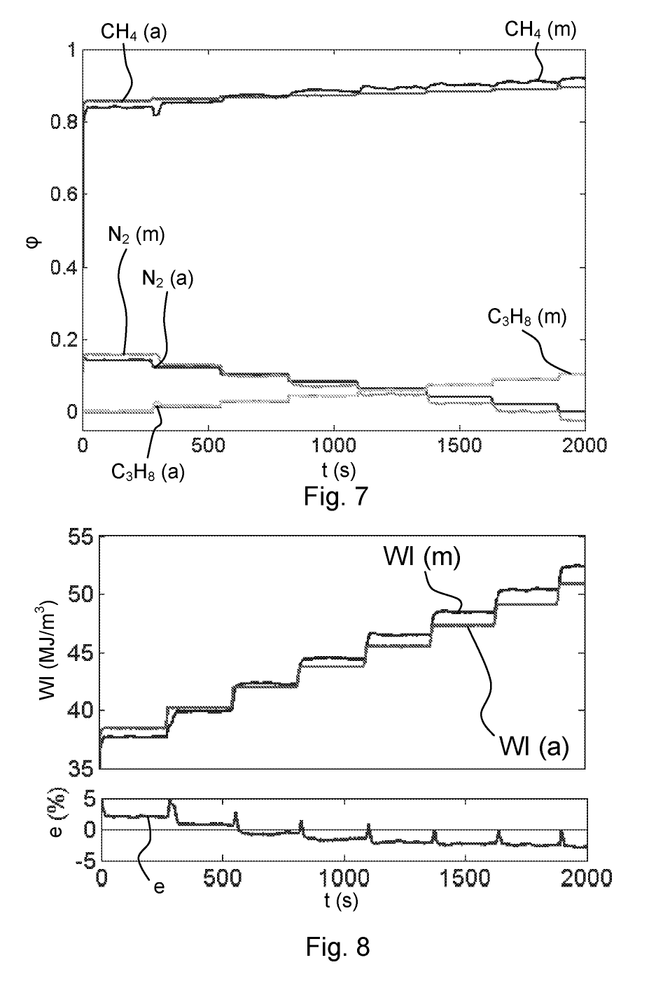

FIG. 7 shows the determination of the composition of a gas mixture comprising CH₄, C₃H₈, and N₂. The values of the fractions measured by a system according to the present invention CH₄ (m), C₃H₈ (m), and N₂ (m) as well as the applied, i.e. actual values CH₄ (a), C₃H₈ (a), and N₂ (a) of the fractions are plotted on the vertical axis against time t, which is plotted on the horizontal axis. Again, the measured values CH₄ (m), C₃H₈ (m), and N₂ (m) follow the actual values CH₄ (a), C₃H₈ (a), and N₂ (a) quickly and accurately. The deviation between the measured values “(m)” and the applied values “(a)” is below five percent.

FIG. 8 shows a further determination of the Wobbe index of a gas mixture comprising CH₄, C₃H₈, and N_{2.} This measurement corresponds to the measurement of FIG. 6, but with the difference that in FIG. 8 the applied Wobbe index WI (a) is given a flatter waveform than in FIG. 6. Again, the determined values WI (m) lie within a five percent deviation with respect to the applied values WI (a).

It will be clear to those skilled in the art that the invention was described above with reference to a few possible embodiments which are regarded as preferable. The invention, however, is by no means limited to these embodiments. Many modifications are possible within the scope of the invention. The protection applied for is defined by the appended claims.

1. A method of determining the fractions of a flowing gaseous medium which consists at least substantially of a known plurality N of known components, which method comprises the following steps:
providing the flowing gaseous medium of which the composition is to be determined,
determining at least N−1 parameters of the gaseous medium provided,
for each of the N known components, providing at least N−1 reference values for each of the determined N−1 quantities,
determining the fraction of each of the known components of the provided gaseous medium through solving of at least N equations, which equations comprise:
at least N−1 equations which describe each determined parameter as a function of the fraction of each of the known parameters of the medium and as a function of the provided reference values for each of the known components of the gaseous medium, and at least one equation which sets the sum of the fractions of each of the known components at least substantially so as to be equal to 100%.

2. A method according to claim 1, wherein the method comprises a step of substantially continuously providing the flowing gaseous medium and of substantially continuously determining the at least N−1 parameters.

3. A method according to claim 1, wherein the known plurality N of known components is equal to at least three or equal to at least four.

4. A method according to claim 1, wherein the method comprises a direct supply of the flowing gaseous medium without any pre-treatment.

5. A method according to claim 1, wherein the steps of determining the parameters and of determining the fractions of the components are repeated at least once.

6. A method according to claim 5, wherein a time interval between two consecutive determinations of fractions lies in a range selected from between 0 and 60 seconds, between 0 and 15 seconds, and between 0 and 5 seconds.

7. A method according to claim 1, wherein at least one of the parameters is chosen from mass flow, density, viscosity, and heat capacity.

8. A method according to claim 7, wherein the density and the heat capacity of the gaseous medium are determined by means of signals from a thermal flow sensor and a flow sensor of the Coriolis type.

9. A method according to claim 1, wherein the equations are solved by a method of least squares.

10. A method according to claim 1, wherein a measure for the calorific value of the flowing gaseous medium is additionally derived from the determined fractions.

11. A method according to claim 10, wherein the Wobbe index of the gaseous medium is additionally derived from the calorific value.

12. A method according to claim 1, comprising a step of controlling the mass flow of the flowing gaseous medium in dependence on the determined fractions thereof.

13. A system for the method according to claim 1 comprising
a flow tube having an inlet and an outlet for supplying and discharging, respectively, the flowing gaseous medium, in particular in a continuous manner, of which medium the composition is to be determined,
sensor means for determining the at least N−1 parameters of the supplied gaseous medium,
a processing unit which is connected to the sensor means, in which the at least N−1 reference values are stored, and which is designed for determining the fraction of each of the known components of the supplied gaseous medium by solving the at least N equations.

14. A system according to claim 13, wherein the sensor means and the processing unit are designed for determining the N−1 parameters and the fractions in a repetitive manner and/or continuously.

15. A system according to claim 14, wherein the system is designed for repeatedly determining the fractions at time intervals that lie in a range selected from between 0 and 60 seconds, between 0 and 15 seconds, and between 0 and 5 seconds.

16. A system according to claim 13, wherein the sensor means comprise at least one of the following: a density sensor, a flow sensor of the Coriolis type, a thermal flow sensor, and/or a pressure sensor.

17. A system according to claim 13, wherein the sensor means comprise at least a thermal flow sensor and a flow sensor of the Coriolis type, and wherein the processing unit is designed for determining the specific heat capacity of the medium on the basis of signals from both the thermal flow sensor and the flow sensor of the Coriolis type.

18. A system according to claim 13, wherein the sensor means comprise at least a flow sensor of the Coriolis type and a pressure sensor, and wherein the processing unit is designed for determining the viscosity of the medium on the basis of signals from both the flow sensor of the Coriolis type and the pressure sensor.

19. A system according to claim 13, wherein the sensor means comprise at least a pressure sensor and a thermal flow sensor, and wherein the processing unit is designed for determining the differential pressure across the thermal flow sensor.

20. A system according to claim 13, further comprising signalling means connected to the processing unit for providing a signal when one of the determined fractions deviates from a standard value.
